(19) United States
(12) Patent Application Publication
(10) Pub. No.: US 2019/0021631 A1
(43) Pub. Date: Jan. 24, 2019

(54) METHOD AND SYSTEM FOR LOCATING MATERIAL ON OR INSIDE A PATIENT'S BODY

(71) Applicant: NEXUS CONTROL SYSTEMS, LLC, PORT WASHINGTON, NY (US)

(72) Inventors: TODD COHEN, PORT WASHINGTON, NY (US); JOHN R. LUBISICH, WEST LINN, OR (US); JOHN DALE MOORE, NEWBERG, OR (US); DIETER MUELLER, NEWBERG, OR (US); MICHAEL MORRISON, TUALATIN, OR (US); GREGORY MORRISON, TUALATIN, OR (US)

(21) Appl. No.: 16/116,287

(22) Filed: Aug. 29, 2018

Related U.S. Application Data
(63) Continuation-in-part of application No. 15/065,595, filed on Mar. 9, 2016.
(60) Provisional application No. 62/130,070, filed on Mar. 9, 2015.
(60) Provisional application No. 62/552,827, filed on Aug. 31, 2017.

(51) Int. Cl. A61B 5/06; A61B 90/00

(52) U.S. Cl. CPC A61B 5/062; A61B 90/39; A61B 2090/395; A61B 2090/3954; A61B 2090/3966; A61B 2090/3925

(57) ABSTRACT

A handheld device for locating a foreign body in or beneath a patient's skin comprises one or more sensors that each generate a magnetic or electromagnetic field and a signal reflecting the location and/or size and/or depth of a foreign body and a processor that receives and processes each signal and generates a signal to an operatively connected display. The display that receives the display signal shows the location and/or size and/or depth of the foreign object.

### CROSS-REFERENCE TO RELATED APPLICATIONS

This application is a continuation-in-part of co-pending U.S. patent application Ser. No. 15/065,595, filed Mar. 9, 2016, which is based upon and claims the benefit of the priority date of U.S. Provisional Patent Application Ser. No. 62/130,070, filed Mar. 9, 2015. This application also is based upon and claims the benefit of the filing date of co-pending U.S. Provisional Patent Application Ser. No. 62/552,827, filed Aug. 31, 2017. Each of these patent applications is incorporated herein in its entirety by reference.

### FIELD OF THE INVENTION

The invention described herein relates to a method and system for detecting or identifying foreign bodies that are on or within the human body. More particularly, this invention provides a system and method for sterile scanning and for marking on the skin the location of metallic and nonmetallic materials or devices that reside on or under the skin in preparation for surgical removal or explantation. In addition, the invention provides a method of scanning a patient prior to wound closure to avoid the complication of a retained foreign body. Further, the sterilizable handheld device can be used post-surgery to identify and locate a retained foreign body.

### BACKGROUND OF THE INVENTION

At present there is no simple and inexpensive sterile medical device and/or system that can be used to scan the human body and detect or identify difficult to find foreign bodies, i.e., metallic or nonmetallic materials, objects, or devices, within the human body that may require removal, revision, replacement, and/or extraction. With some larger devices, such as implantable defibrillators, it is easy to find such a subcutaneous device because of its size, mass, and location of placement. Up until recently, smaller devices, such as a pacemaker, could usually be identified. However, newer systems and technology are often so small that location during surgery—especially after sterile draping of the patient—may be difficult. Operators have experienced difficulty finding a paper clip size subcutaneous metallic loop recorder and can inadvertently hit an artery, which could contribute to bleeding. This can occur even with the presence of X-ray equipment such as a fluoroscope and other surgical exploratory tools. Thus, there is a need to precisely locate a foreign body, material, or device and mark the skin, prior to the surgical exploration, and particularly in a manner which does not break sterility during the surgical procedure.

Currently, hidden medical implants and other metal objects can be identified via X-ray methodology prior to surgical removal, revision, extraction, explantation, or the like. This methodology exposes the patient to potentially harmful X-rays. In addition, the operator has to wear heavy X-ray shielding equipment (for example, lead aprons) prior to or during the surgery. In addition, this methodology is often not precise, and a metal or radio-opaque tool also needs to be utilized to help locate the metallic object beneath the X-ray beam.

Alternatively, skin scars and marks from prior wounds can help to show the initial location in which a metallic or nonmetallic material, object, or device may have traversed the skin prior to its final resting site within a human body. Blindly opening such a site and exploring for the metallic or nonmetallic material, object, or device can be frustrating and has the potential for complications.

Therefore, there is also a need for a simple, inexpensive, and sterile non-X-ray system that could be used before, and even during, surgery to help detect or localize a foreign body, that is, a metallic or nonmetallic material, object, or device, and sterilely mark a patient's skin. In addition, there is a need to have such system be either disposable or resterilizable for such a surgical procedure. There also is a need to have the system optionally include a sterile marker either attached to or provided with the scanning metal medical device to best mark the location of the device prior to any skin puncture or incision.

Retained foreign bodies following surgery are a source of medical errors and may result in a medical malpractice lawsuit. There is a need for a simple scanning device to detect retained foreign bodies (metal and otherwise). A simple handheld scanner that could detect such retained foreign bodies could be used before closing a surgical wound and especially prior to the patient's leaving the operative suite to prevent additional surgeries by identifying a retained foreign body and facilitating removal of the unintended material(s). Then, the wound/incision could be safely and surgically closed with peace of mind that there is no retained foreign body.

### OBJECTS OF THE INVENTION

It is an object of the invention to provide a handheld sterile system for detecting, locating, or identifying hidden materials, objects, or devices within the human body prior to surgical exploration, removal, revision, or replacement.

It is also an object of the invention to provide a method for detecting, locating, or identifying hidden materials, objects or devices within the human body prior to surgical exploration, removal, revision, or replacement.

It is a further object of the invention to provide a system in which a sterile medical device and marker are used to identify the location of a hidden foreign body prior to or during a surgical procedure.

It is a yet further object of the invention to provide a system in which the sterile medical device comprises a power supply, object detector, monitor/alarm system, display, and a plastic or rubber, waterproof, or water resistant casing.

It is a yet further object of the invention to provide a system wherein a handheld medical device can determine the location of metallic materials, objects, or devices either on or under the skin.

It is a yet further object of the invention to provide a system wherein a handheld medical device can determine the location and/or size and/or depth of a subcutaneous foreign body.

It is a yet further object of the invention to provide a system wherein a handheld medical device has a main component that can be re-sterilized.

It is a yet further object of the invention to provide a system wherein a handheld medical device for determining the location and/or size and/or depth of a subcutaneous foreign body has one or more sensors.

It is a yet further object of the invention to provide a system wherein a disposable, handheld medical device for determining the location and/or size and/or depth of a subcutaneous foreign body has two or three sensors.

It is a yet further object of the invention to provide a system wherein a handheld medical device for determining the location and/or size and/or depth of a subcutaneous foreign body has a disposable component capable of sterile marking.

It is a yet further object of the invention to provide a system wherein a handheld medical device can determine the location of metallic or nonmetallic materials, objects, or devices either on or under the skin.

It is a yet further object of the invention to provide a system wherein a handheld medical device can determine the location of ferrous or nonferrous materials, objects, or devices.

It is a yet further object of the invention to provide a system in which the handheld medical device is disposable.

It is a yet further object of the invention to provide a system in which the handheld medical device is sterilizable or resterilizable.

It is a yet further object of the invention to provide a system in which the materials, objects, or devices to be identified and/or located and/or detected are metallic or nonmetallic.

It is a yet further object of the invention to provide a sterile handheld medical system comprising means for scanning and detecting or locating a foreign body hidden in or underneath a patient's skin based on an audible and/or visual feedback system.

It is a yet further object of the invention to provide a sterile handheld medical system also comprising means for marking the location of the foreign body within a patient or on the patient's skin.

It is a yet further object of the invention to provide a sterile handheld medical system also comprising a tool for removing a foreign body beneath a patient's skin from the patient.

It is a yet further object of the invention to provide a sterile method for scanning and detecting or locating a foreign body hidden in or underneath a patient's skin based on an audible and/or visual feedback system, optionally marking the location of the foreign body on the patient's skin, and then removing the foreign body.

It is a yet further object of the invention to provide a system which comprises a medical scanner or locator, a skin puncturing tool, a digging tool, and an object grabber to securely grab onto a foreign body such that it can be retracted and the foreign body is removed from the patient's body.

It is a yet further object of the invention to provide a system wherein the digging tool and grabber contain a locating element to hone in on and precisely locate a foreign object.

It is a yet further object of the invention to provide a handheld sterile medical device and system which can scan and detect or localize, that is, locate, a foreign body and mark the location of the foreign body hidden within a patient's body prior to surgical exploration, removal, revision, or replacement.

It is a yet further object of the invention to provide a device or system wherein foreign bodies are detected or located by a method applied at or near the surface of the patient's skin.

It is a yet further object of the invention to provide a method, device or system which comprises ultrasound technology, changes in resistance, resistivity, inductance, or the dielectric current, changes in the magnetic field, changes in the electrical current, optical light changes, thermal changes, and/or radiant heat, and/or use of a radiofrequency sensor.

It is a yet further object of the invention to provide a device or system which comprises technology for determining the depth of one or more foreign bodies within a patient's body.

It is a yet further object of the invention to provide a device or system which comprises technology for determining the location and/or size and/or depth of a foreign object within a patient's body.

It is a yet further object of the invention to provide a handheld sterile system for detecting or identifying the location and/or size and/or depth of a foreign body hidden within a patient's body prior to surgical wound closure, following surgical wound closure, or after surgery.

It is a yet further object of the invention to provide a sterile method for identifying and locating a foreign body hidden within a patient's body prior to surgical wound closure, following surgical wound closure, or after surgery.

It is a yet further object of the invention to provide a system for identifying and locating both foreign objects and blood vessels to increase safety of a surgical procedure by avoiding inadvertent damage to a significant blood vessel that might cause bleeding or other complications.

These and other objects of the invention will become more apparent from the description, drawings, and claims below.

### SUMMARY OF THE INVENTION

Described herein is a precise method and system for locating any foreign bodies, i.e., materials, objects, or devices, metallic or nonmetallic, hidden on or beneath a patient's skin. More particularly, a system and a method identifies and optionally marks a patient's skin for surgical exploration, identification, removal, revision, and replacement of metallic and nonmetallic foreign bodies located on and/or under the skin. In addition, the scanner could be used to assure that there is no retained foreign body following surgery and prior to wound closure.

Scanners exist for detecting or identifying structures comprised of metallic or non-metallic materials that may be hidden beneath a wall. Devices that are typically available in hardware stores, known as stud finders, measure changes in dielectric current and can identify the presence of wood beams or studs beneath a wall. Such stud finders can be tuned and provide feedback to the user to locate wooden beams or studs. In addition, metal scanners can be used to locate both ferrous and nonferrous metals which might be hidden beneath a wall. For example, Zircon makes hardware tools that function as a stud finder, a metal scanner, or both a stud finder and a metal scanner. Zircon's stud finder device can identify the edges of wooden beams beneath a wall, and Zircon's metal scanner can identify nails, screws, and pipes that are hidden behind a wall. Nonferrous materials such as copper that are up to two inches deep and ferrous materials such as steel that are up to four inches deep can be identified in this way. The Zircon devices can find (1) studs, (2) metal objects, or (3) both studs and metal objects.

The Zircon system uses a sensor with an inductive coil that generates a magnetic field to identify the hidden object. A series of light indicators (LEDs) along with an audible sound reflect the proximity of a hidden object. Alternatively, a display monitor can be used to also indicate the proximity of the hidden object.

One example of a device used by carpenters is the Zircon METALLISCANNER® M40 stud finder. This is a handheld carpentry tool, which can detect metal behind a wall (ferrous and nonferrous). It is approximately 8 inches long, handheld, and powered by a 9 volt battery. It uses a series of red lights (LEDs) as well as an audible tone to alert the user that a metal object has been located. See, for example, Zircon's U.S. Pat. No. 7,812,722, incorporated herein by reference in its entirety, which describes a hardware store device that can use two modes to detect metal.

The invention herein provides a system and method for sterile scanning and marking on or of a patient's skin the location of metallic or nonmetallic materials that reside on or under the patient's skin. Such materials, which may generally be known as “foreign bodies” or “foreign objects,” that is, materials, objects, or devices that are not naturally occurring in a patient's body, include, for example, an implantable device such as a pacemaker, an implantable loop recorder, an implantable physiologic monitor, an implantable sensor, an implantable tracker, implantable communicators, an implantable prostheses, implantable GPS systems, pumps, monitors, pacemakers, defibrillators, drug delivery pumps, pins, screws, or other hardware, retained needles or any equipment left from surgery, or an advertent or inadvertent implantable device or material of any sort. In addition, the materials may also include non-implantable materials such as a bullet or shrapnel, which may require surgery for removal or explantation. The system could also be used to scan a patient prior to surgical wound closure to avoid the problem of a retained foreign body and the need for a second surgical procedure to find and remove the retained foreign body. This novel sterile handheld scanner could also be used in the retained foreign body cases to identify the retained foreign material, localize it, and remove it accordingly.

In an embodiment of the invention, a handheld sterile, sterilizable, or resterilizable system is capable of detecting, locating, or identifying hidden foreign bodies within a patient's body prior to surgical exploration, removal, revision, or replacement.

In another embodiment of a method of the invention, a handheld sterile, sterilizable, or resterilizable system is used to detect, locate, or identify subcutaneous hidden foreign objects within a patient's body prior to surgical exploration, removal, revision, or replacement.

In another embodiment of the invention, a sterile, sterilizable, or resterilizable medical device and marker are used to detect, locate, determine, or identify the location and/or size and/or depth of a subcutaneous foreign object prior to a surgical procedure.

In another embodiment of the invention, the sterile, sterilizable, or resterilizable medical device comprises a power supply, object detector, monitor/alarm system, and a plastic or rubber, waterproof or water resistant casing.

In another embodiment of the invention, the handheld medical device can detect and determine the location of metal materials, objects, or devices either on or under a patient's skin.

In another embodiment of the invention, in a handheld medical system for detecting a location of a foreign object within a patient's body based upon audible and/or visual feedback prior to surgical exploration, removal, revision, or replacement, the system comprises a power supply, a metal scanner or object detector, and an audible and/or visual feedback system.

In another embodiment of the invention, a system is capable of marking the location of the foreign object prior to a surgical procedure.

In another embodiment of the invention, a system can detect the location of a metal or nonmetal foreign object either on or under skin of a patient.

In another embodiment of the invention, a foreign object is ferrous or nonferrous.

In another embodiment of the invention, a system is capable of detecting the location of more than one foreign object.

In another embodiment of the invention, a system comprises a tool for finding and then removing a foreign object.

In another embodiment of the invention, a system comprises a skin puncturing tool, a digging tool, and an object grabber to securely grab a foreign object so that it can be retracted and removed from a patient's body.

In another embodiment of the invention, a digging tool and object grabber comprise a locating element to hone in on and precisely locate a foreign object.

In another embodiment of the invention, the location of a foreign object is detected by ultrasound technology, changes in resistance, resistivity, or the dielectric current, changes in the magnetic field, changes in the electrical current, optical light changes, thermal changes and/or radiant heat, and/or use of a radiofrequency sensor.

In another embodiment of the invention, a system comprises a monitor/alarm system and/or a plastic or rubber waterproof or water resistant casing.

In another embodiment of the invention, a system comprises technology for determining the location, size, and/or depth of a foreign object within a patient's body.

In another embodiment of the invention, a system identifies foreign objects located within a human body prior to surgical wound closure, following surgical wound closure, or after surgery to identify and locate a retained foreign body.

In another embodiment of the invention, a system identifies and determines the locations of both foreign objects and blood vessels to increase safety of a surgical procedure by avoiding inadvertent damage to a significant blood vessel that might cause bleeding or other complications.

In another embodiment of the invention, a system is capable of determining the location of a foreign object within a patient's body and of finding and removing the foreign object.

In another embodiment of the invention, a system is capable of detecting the location of a foreign object within a patient's body, marking the location of the foreign object, and finding and removing the foreign object.

In another embodiment of the invention, the handheld medical device can determine the location of a ferrous or nonferrous foreign body.

In another embodiment of the invention, one or more components of the medical device are sterile, sterilizable, or resterilizable and/or disposable.

In another embodiment of the invention, the materials, objects, or devices to be detected are made of a material other than metal.

In another embodiment of a system of the invention, the system is capable of scanning and determining the location of a material, object, or device hidden in or underneath a patient's skin based on an audible and/or visual feedback system.

In another embodiment of a system of the invention, the system is capable of sterile marking on a patient's skin the location of a material, object, or device hidden in or underneath the patient's skin.

In another embodiment of a system of the invention, the system comprises a tool capable of removing from a patient a material, object, or device hidden in or underneath the patient's skin.

In another embodiment of the invention, a handheld medical system comprises means for scanning and determining the location of a material, object, or device hidden in or underneath a patient's skin, marking the location of the material, object, or device on the skin, and finding and then removing the material, object, or device, based on an audible and/or visual feedback system.

In another embodiment of the invention, a method comprises scanning and detecting or determining the location of a foreign body hidden in or underneath a patient's skin based on an audible and/or visual feedback system, optionally marking the location of said foreign body on the skin, and then removing the foreign body.

In another embodiment of the invention, a system comprises a medical scanner or locator, a skin puncturing tool, a digging tool, and a foreign body grabber to securely grab onto the foreign body such that is can be retracted and the foreign body is removed from the human body.

In another embodiment of the invention, the digging tool and grabber contain a locating element to hone in on and precisely determine the location of a foreign object hidden in or underneath a patient's skin.

In another embodiment of the invention, a handheld sterile medical device and system can scan and detect or localize, that is, determine the location of, a foreign body hidden in or underneath a patient's skin and mark the foreign body prior to surgical exploration, removal, revision, or replacement.

In another embodiment of the invention, the location of foreign bodies hidden in or underneath a patient's skin is determined by a method and device applied at or near the surface of the patient's skin.

In another embodiment of the invention, a foreign body hidden in or underneath a patient's skin is detected, located, or identified by ultrasound technology, inductance, changes in resistance, resistivity, or the dielectric current, changes in the magnetic field, changes in the electrical current, optical light changes, thermal changes, and/or radiant heat, and/or use of a radiofrequency sensor.

In another embodiment of the invention, a device or system comprises technology for determining the depth underneath a patient's skin of a foreign body.

In another embodiment of the invention, a device or system comprises technology for determining the location and/or size and/or depth of a foreign object within a patient's body.

In another embodiment of the invention, a foreign object within a patient's body is detected, located, or identified prior to surgical wound closure, following surgical wound closure, or after surgery, to identify and determine the location of a retained foreign body.

In another embodiment of the invention, one or more foreign objects or devices and blood vessels underneath a patient's skin are detected, located, or identified to increase safety of a surgical procedure by avoiding inadvertent damage to a significant blood vessel that might cause bleeding or other complications.

In one embodiment of the invention, a scanning device could comprise a rubber or plastic sterile waterproof or water resistant shielded device which encompasses (1) a power supply, (2) one or more sensors/detectors, and (3) monitor and/or alarm (visual and/or audio).

In another embodiment of a medical scanning device of the invention, a sterile marker is included together with the scanning device to mark a patient's skin in sterile fashion at the location at which implantable or implanted material, an object, or a device hidden in or underneath a patient's skin.

The scanning methods may include, but are not limited to, methods applied at or near the surface of a patient's skin, such as ultrasound technology, changes in inductance, resistance, resistivity, or the dielectric current, changes in the magnetic field, optical light changes, thermal temperature changes, and/or radiant heat.

In another embodiment of the invention, a light and/or sterile marker is contained within a medical scanning device such that the light would shine at the location where the detection signal is the strongest. Alternatively, the sterile marker could be advanced from a retracted position from the device to mark the skin in sterile fashion and then retracted when removed.

In another embodiment of the invention, a surgical tool is attached to a location device such that (1) the patient's skin could be anesthetized and/or (2) the patient's skin could be punctured, and/or (3) the surgical tool could advance underneath the patient's skin towards an indentifiable subcutaneous object or device, that is, a foreign body, and/or (4) the surgical tool would create a louder signal and/or visual cue helping the location device precisely locate the foreign body, and/or (5) a grabbing tool could grab such foreign body and securely hold it in place, and/or (6) said grabbed foreign body could be retracted into a removal tool and removed out of the patient to remove said foreign body.

In another embodiment of the invention, the scanner device may have a protective boot or sheath to keep the device sterile and clean. Such a protective boot or sheath may be flexible, rigid, or a combination thereof, preferably from a sterile or sterilizable material such as a polyurethane, polyethylene, or other polymer or copolymer.

In another embodiment of the invention, the location of an implanted or subcutaneous foreign body can be marked by, for example, a colored sticker with or without writing or symbols placed on a patient's skin, marking on the skin from a marker comprising indelible ink, or a radiopaque sticker.

In another embodiment of the invention, a method and system for identifying and locating both foreign bodies and blood vessels underneath a patient's skin increases the safety of a surgical procedure by avoiding inadvertent damage to a significant blood vessel that might cause bleeding or other complications.

In another embodiment of the invention, a scanning device comprises technology or structure to indicate the depth of an implanted or subcutaneous foreign body. For example, the scanning device may comprise a telescoping rod or member that calibrates the edge of the electromagnetic field, to determine depth with almost pinpoint accuracy. If the telescoping rod is graduated, it could provide an estimate on the depth under the surface of the skin. The shorter the rod, the greater the depth. This will ensure the smallest incision size and allow a doctor to find an object or device in the most expedient manner possible because the doctor will have an estimate of the depth below the epidermis.

The invention herein is directed to a handheld, sterile, sterilizable, or resterilized device. It is within the scope of the invention that the device could be similarly sized unit or a larger device attached to a console or other structure.

In another embodiment of the invention, a handheld medical scanning system for determining location and/or size and/or depth of a foreign body on or beneath a patient's skin, comprises one or more sensors and a processor to provide precise location and/or size and/or depth information.

In another embodiment of the invention, a handheld medical scanning device uses input from a plurality of sensors to triangulate the precise location and/or size and/or depth of a foreign body imbedded in or below the patient's skin.

In another embodiment of the invention, a handheld medical scanning device comprises a processor that provides audible and/or visible feedback.

In another embodiment of the invention, a handheld medical scanning comprises a power supply, object detector, monitor/alarm system, microprocessor, display, and a plastic or rubber waterproof or water resistant casing.

In another embodiment of the invention, a handheld medical scanning device can detect a foreign body prior to surgical exploration, removal, revision, or replacement.

In another embodiment of the invention, a foreign body is an implantable medical device.

In another embodiment of the invention, the implantable medical device is a medical diagnostic device selected from the group consisting of a heart rhythm recorder; an implantable loop recorder; a glucose, temperature, respiration, heart failure, impedence, pH, or physiologic body detector or recorder; or another physiologic monitor.

In another embodiment of the invention, the implantable medical device is a medical therapeutic device selected from the group consisting of implantable pacemakers, implantable defibrillators, implantable heart failure devices, drug delivery devices or pumps, and any other medical therapeutic device.

In another embodiment of the invention, a diagnostic and therapeutic device comprises a system as described herein and a feedback loop to diagnose and provide treatment to a patient.

In another embodiment of the invention, the foreign body is a GPS tracker, a personal and/or medical identification recorder, a payment recorder, a health information recorder, a communication system, or any combination thereof.

In another embodiment of the invention, each sensor generates a magnetic or electromagnetic field.

In another embodiment of the invention, each sensor generates and/or senses ultrasound radiation.

In another embodiment of the invention, each sensor extends longitudinally.

In another embodiment of the invention, there are two or more longitudinally extending sensors that extend in parallel.

In another embodiment of the invention, a handheld medical scanning device comprises one or more components that are capable of sterilely marking the patient's skin with respect to location of the foreign body prior to removal and/or replacement of the foreign body.

In another embodiment of the invention, a marking component is disposable or resterilizable.

In another embodiment of the invention, a marking component activates the handheld scanning device.

In another embodiment of the invention, the handheld scanning device is resterilizable, reprocessable, or both resterilizable and reprocessable.

In another embodiment of the invention, a handheld medical scanning device also comprises a tool for finding and then removing a foreign body.

In another embodiment of the invention, a handheld medical scanning device which comprises a handheld scanner and/or detector, a skin puncturing tool, a digging tool, and/or a grabber to securely grab a foreign body so that it can be retracted and removed from the patient's body.

In another embodiment of the invention, a handheld medical scanning which comprises a skin closure system to medically close an opening in the patient's skin.

In another embodiment of the invention, in a handheld medical scanning device a digging tool and/or grabber comprise a locating element to hone in on and precisely locate the foreign object.

In another embodiment of the invention, a handheld medical scanning device can detect, remove, and replace an implantable medical device.

In another embodiment of the invention, a foreign body is detected using one or more sensors that detect changes with respect to ultrasound, resistance, resistivity, dielectric current, the magnetic field, electrical current, optical light, temperature, radiant heat, and/or radiofrequency.

In another embodiment of the invention, a handheld medical scanning device is useful for identifying objects hidden within the human body prior to surgical wound closure, following surgical wound closure, or after surgery to identify and locate a retained foreign body.

In another embodiment of the invention, a handheld medical scanning device is useful for identifying and locating both foreign objects and blood vessels to increase safety of a surgical procedure by avoiding inadvertent damage to a significant blood vessel that might cause bleeding or other complications.

In another embodiment of the invention, a method for locating and optionally removing a foreign body within a patient's body on or beneath the patient's skin prior to surgical exploration, removal, revision, or replacement, comprises using a medical system as described herein to determine the location and/or size and/or depth of the foreign body.

In another embodiment of a method of the invention, the location of a foreign body is marked on the patient's skin.

In another embodiment of a method of the invention, a tool forming part of the medical system is used to extract a foreign body.

In another embodiment of a method of the invention, foreign bodies are located by a method applied at or near a surface of the patient's skin.

In another embodiment of the invention, a handheld device for locating a foreign body in or beneath a patient's skin, comprises

one or more sensors that each generate a magnetic field and a signal reflecting the location and/or size and/or depth of a foreign body;

a processor or microprocessor that receives and processes each signal and generates a signal to an operatively connected display; and

a display that receives the display signal and shows the location and/or size, and/or depth of the foreign object.

In another embodiment of the invention, there are two or three sensors.

In another embodiment of the invention, the device has a base and there are three sensors equidistantly positioned around the base.

### BRIEF DESCRIPTION OF THE DRAWINGS

FIG. 1 is a representation of an embodiment of the invention positioned at a distance from an object to be detected or located. The telescoping rod is retracted;

FIG. 2 is a representation of the embodiment shown in FIG. 1 where the telescoping rod has been extended;

FIG. 3 is a representation of the embodiment shown in FIG. 2 where a sterile barrier has been attached to the telescoping rod;

FIG. 4 is a representation of another embodiment of the invention;

FIG. 5 is a perspective view of the inner workings of the embodiment shown in FIG. 4; and

FIG. 6 is a block diagram of the embodiment shown in FIGS. 4 and 5.

### DETAILED DESCRIPTION OF THE INVENTION

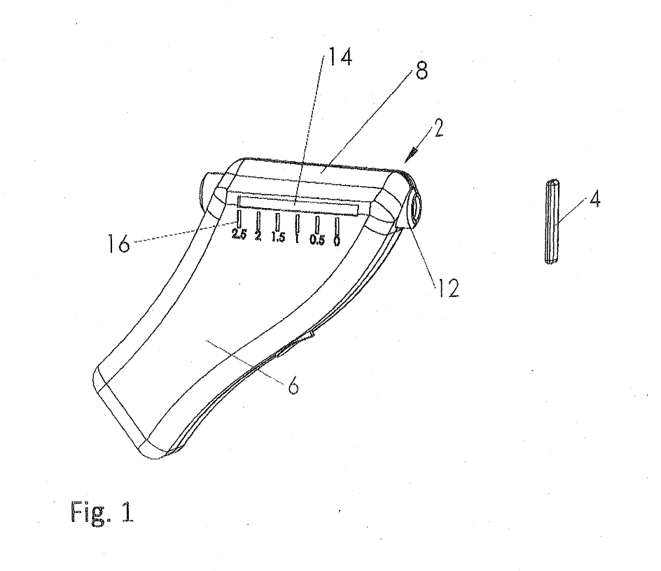

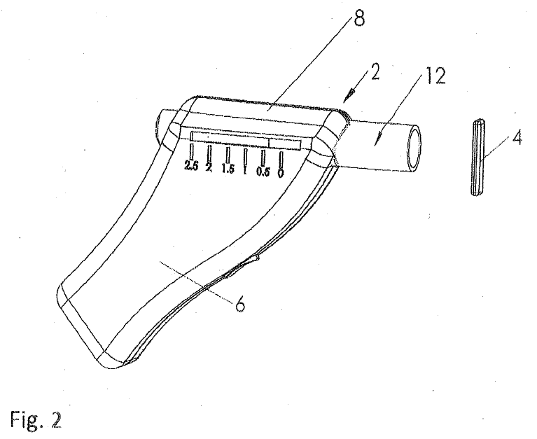

The invention can perhaps be better appreciated by reference to the drawings. In FIG. 1, a device 2 is positioned at a distance from an object 4 to be detected. Location device 2 comprises a grippable handle 6 attached to an upper portion 8. Upper portion 8 preferably comprises a telescoping rod 12, shown retracted in FIG. 1. In FIG. 2, telescoping rod 12 is extended. Telescoping rod comprises a cylindrical sensor that generates electromagnetic waves or energy.

Upper portion 8 has a lever or other mechanism (not shown) for advancing telescoping rod 12. Upper portion 8 comprises a window 14 with markings 16 that correspond to and measure the extent that telescoping rod 12 is extended.

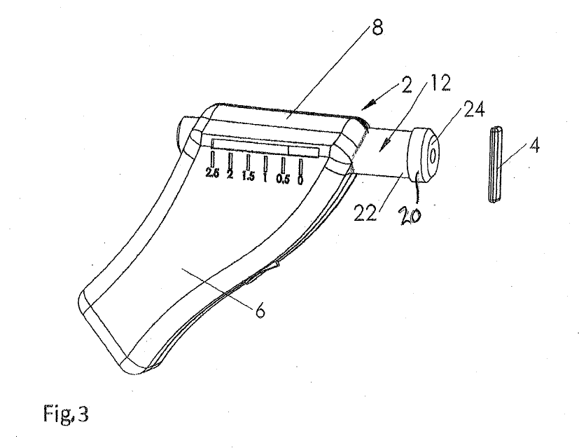

In FIG. 3, telescoping rod 12 has a sterile barrier or tip 20 positioned on its distal end 22. Barrier or tip 20 has a sticker or marker 24 for indicating the location of a subcutaneous foreign body when sterile barrier or tip 20 is pressed against a patient's skin (not shown). Sticker or mark 24 may be, for example, a flat circle or annular-shaped substrate with an adhesive surface or powder or ink. Alternatively, tip 20 comprises tape with non-toxic powder or ink that marks a patient's skin to show location of a subcutaneous object or device.

Barrier or tip 20 may comprise a nose cone that functions as an activator. When the nose cone is snapped into place onto telescoping rod 12 or upper portion 8, it either completes a circuit or provides a code, or something functionally similar, to enable the functionality of the device. Without the nose cone tip 20 with the marker, the device is inoperable. Thus, nose cone 20 can be a critical accessory to the functionality of the device.

Object 4 shown in FIGS. 1 to 3 is a foreign body such as a LINQ™ Insertable Cardiac Monitor, available from Medtronic, or a BioMonitor® implantable loop recorder available from Biotronik. When a device such as the LINQ or BioMonitor device is implanted, its depth is unknown. Telescoping rod 12 can be used to ascertain position and depth. Telescoping rod 12 will preferably be designed so that it is paired to the type of device it is locating. This way a device with a stronger reflective field will have a rod that is longer, and a device with a weak reflective field will have a shorter rod. The paired rod will then allow a user to estimate approximate depth by locating the device center and then extend the rod until the device is no longer detected, thus providing an approximate depth in the tissue. The rod will be graduated to provide the user with an approximate depth.

The embodiment of the invention represented in FIGS. 1-3 comprises a single sensor or antenna (not shown) in telescoping rod 12. The sensor could be a tightly wound cylindrical inductive coil having a longitudinally extending core, which inductive coil generates a magnetic or electromagnetic field. For example, the sensor could comprise an air core or a solid or annular core from about 1 to three inches long with an outer diameter of from about 0.3 to 0.5 inches having a coil with from approximately 40 to 120 turns. Alternatively, the sensor could be a ferrite rod or annulus or a rod or annulus of another material that will generate a magnetic field.

It is within the scope of the invention that a handheld scanner may comprise one or more sensors, preferably two, three, or four sensors, for example, including three equidistantly positioned sensors. When there are two or more sensors, the sensors are preferably positioned to have substantially parallel, longitudinally extending cores. The sensors will extend in the direction of an expected device or foreign body, essentially perpendicular to the patient's skin surface above the device or foreign body. Information from the sensor or sensors is transmitted to a processor or microprocessor to determine the location and/or size and/or depth of a foreign body.

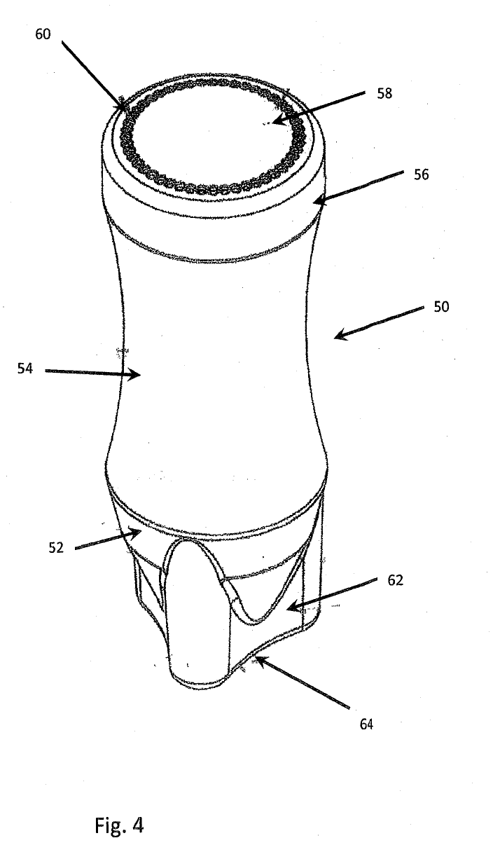

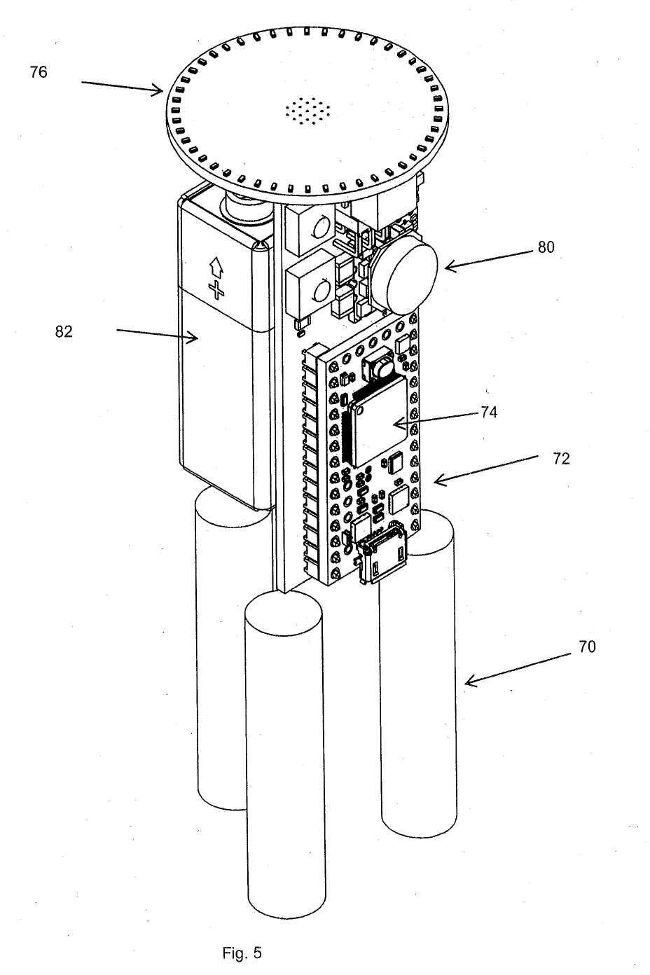

In the embodiment of the invention shown in FIGS. 4 and 5, a handheld scanner 50 comprises three sensor components, sensors, or antennas 70 arranged within the distal portion 52 of scanner body 54. Each sensor component 70 comprises a coil or rod surrounding or having a core. The sensor components 70 are preferably spaced equidistantly from one another, such as to form, in cross-section, an equilateral triangle, where the respective sensors and their cores or rods are substantially parallel to one another. The sensor components 70 work in concert with each other to allow the scanner 50 to identify the location and/or size and/or depth of an object or device on a plane relative to scanner 50 which will sit at the origin of the plane. In a practical sense, a scanner 50 with three equidistantly arranged sensors will be best at establishing or determining location of a subcutaneous object. Approximate size may be determined by moving scanner 50 in different directions in a plane to observe changes in image or signal strength. Similarly, determining depth, if possible, may be a function of the operator's experience with the scanner 50 and the signals or images produced. Determining depth would be better if a forth sensor is arranged to give a different dimension.

Scanner body 54 has a proximal end 56 that comprises a screen or display 58. Screen or display 58 displays information regarding an object or device, that is, a foreign body (not shown), sensed by sensor components 70. For example, screen or display 58 may comprise LED lights or arrows 60 that illuminate to reflect the location and/or size and/or depth, or two or more thereof, of a foreign body (not shown) beneath a patient's skin (not shown). Scanner 50 notifies the user when it is directly above the object or device.

More particularly, each sensor component 70 may be a tightly wound cylindrical inductive coil having a longitudinally extending core, which inductive coil generates a magnetic field. For example, each sensor component 70 could comprise an air core or a solid or annular core from about 1 to two inches long with an outer diameter of from about 0.3 to 0.5 inches having a coil with from approximately 40 to 80 turns. Alternatively, a sensor component 70 could be a ferrite rod or annulus or a rod or annulus of another material that will generate a magnetic field.

Preferably scanner 50 has three sensors 70 that are used to locate a foreign body (not shown) below the surface of a patient's skin using triangulation. The three sensors 70 work in concert with each other to allow scanner 50 to identify the location of the foreign body on a plane relative to scanner 50, which will be positioned at the origin of the plane. Optionally the three sensors will also be capable of indicating the size and/or depth of the foreign body. Scanner 50 uses an array of LEDs or arrows in display 58 to direct the user toward the foreign body detected. Scanner 50 notifies the user when scanner 50 is directly above the foreign body.

In one embodiment, scanner 50 may have a fourth sensor (not shown) in scanner body 54 to allow feedback in the z-axis or vertical axis. This would facilitate detecting a foreign body on the xy plane and provide more accurate information regarding the depth of the foreign body in the z-plane.

Scanner 50 has a disposable base 62. Base 62 can be supplied sterile and may contain an apparatus 64 for marking a surface, such as black indelible marker, incorporated into base 62. When the location of an object is detected, the user touches the device to the patient's skin, marking it, or presses a button (not shown) near base 62 which mechanically lowers the marker, and marks the location on the surface of the patient's skin. Base 62 can be used to turn “on” the device and to calibrate the sensitivity of the device. The calibration would be necessary for accurate representation of the depth of an object in the vertical axis. Base 62 can mechanically actuate a strip or contain an RFID chip to allow scanner 50 to identify the calibration settings and authenticity of base 62. Base 62 also acts as a barrier between scanner 50 and the surface of the patient's skin (not shown). Preferably base 62 comprises a marker/nose cone element (not shown) that snaps into place to optionally activate scanner 50.

In the interior view of scanner 50 shown in FIG. 5, each sensor 70 is operatively connected to a control module 72 that comprises a processor 74. Signals from each sensor 70 are received in processor 74, which computes or determines the location, size, or depth, or a combination thereof, of a subcutaneous foreign body (not shown). Control module 72 is operatively connected to a screen or display 76 that displays information, preferably in the form and intensity of lights. Optionally there could be a wired or wireless connection (not shown) to display the information on a separate monitor or mobile device.

Control module 72 is operatively connected to an audio module 80 that generates audio signals that indicate depth or location, or both, to an operator. Power can be supplied by one or more batteries 82, preferably rechargeable, or an electric cord (not shown).

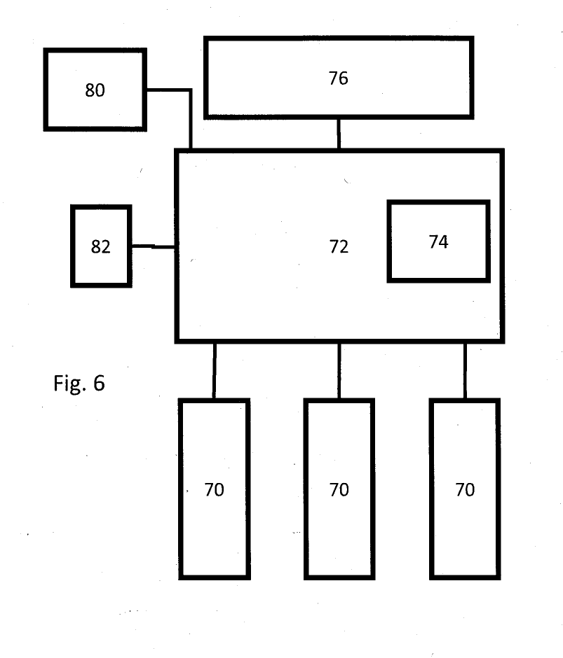

FIG. 6 is a modified block diagram where each sensor 70 is connected to a control module 72 that comprises a processor 74. Signals from each sensor 70 are received in processor 74, which computes or determines the location, size, or depth, or a combination thereof, of a subcutaneous foreign body (not shown). Control module 72 is operatively connected to a screen or display 76 that displays information, preferably in the form and intensity of lights.

Control module 76 is optionally and preferably, operatively connected to an audio module 80 that generates audio signals that indicate depth or location, or both, to an operator. Power can be supplied by one or more batteries 82, preferably rechargeable, or an electric cord (not shown).

The handheld scanning device of the invention device is resterilizable, reprocessable, or both resterilizable and reprocessable. Reprocessing includes, for example, opening the casing of the device, removing and replacing a battery, resetting a computer chip and/or timer, resealing the casing, resterilizing the device, and repackaging the device.

The system described and claimed herein may include a skin closure device for opposing the skin opening and medically sealing the opening. A variation of the system claimed herein may comprise a single device that detects a foreign body, sterilely marks the patient's skin, nicks the skin, and provides a digging tool to be advanced toward the foreign body, using a detection modality. The system engages the foreign body, removes it, and then optionally provides antibiotics and hemostasis inside the retrieval site. The retrieval site can be automatically or semiautomatically closed with a sterile bandage.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

What is claimed:

1. A handheld medical scanning system for determining a location of a foreign body on or beneath a patient's skin, which comprises:
one or more sensors to sense location information;
a microprocessor to process the sensed information; and
an audible monitor and/or visual display to provide audible and/or visual feedback to display the location of the foreign body.

2. The handheld medical scanning system of claim 1, wherein input from a plurality of sensors is processed to triangulate the precise location and/or size and/or depth of the foreign body.

3. The handheld medical scanning system of claim 1 which comprises a power supply, object detector, monitor/alarm system, and a plastic or rubber waterproof or water resistant casing.

4. The handheld medical scanning system of claim 1 which is capable of determining the location of a foreign body prior to a surgical procedure selected from the group consisting of surgical exploration, removal, revision, and replacement.

5. The handheld medical scanning system of claim 1, wherein the foreign body is an implantable medical device, a medical diagnostic device, a medical therapeutic device, a GPS tracker, a personal and/or medical identification recorder, a payment recorder, a health information recorder, a communication system, or any combination thereof.

6. The handheld medical scanning system of claim 1, wherein each sensor generates a magnetic or electromagnetic field or generates and/or senses ultrasound radiation.

7. The handheld medical scanning system of claim 1 which comprises one or more components that are capable of sterilely marking the patient's skin with respect to location of the foreign body prior to removal and/or replacement of the foreign body.

8. The handheld medical scanning system of claim 1 which also comprises a tool for finding and then removing the foreign body.

9. The handheld medical scanning system of claim 8 which comprises a handheld scanner and/or detector, a skin puncturing tool, a digging tool, and/or a grabber to securely grab a foreign body so that it can be retracted and removed from the patient's body.

10. The handheld medical scanning system of claim 9, wherein the digging tool and/or grabber comprise a locating element to hone in on and precisely locate the foreign body.

11. The handheld medical scanning system of claim 1, wherein one or more sensors detect changes with respect to ultrasound, resistance, resistivity, dielectric current, the magnetic field, electrical current, optical light, temperature, radiant heat, and/or radiofrequency.

12. The handheld medical scanning system of claim 1 which is capable of identifying and/or locating objects hidden within a human body prior to surgical wound closure, following surgical wound closure, or after surgery to identify and locate a retained foreign body.

13. The handheld medical scanning system of claim 1 which is useful for identifying and locating both foreign objects and blood vessels to increase safety of a surgical procedure by avoiding inadvertent damage to a significant blood vessel that might cause bleeding or other complications.

14. The handheld medical scanning system of claim 1, wherein the one or more sensors extend longitudinally.

15. The handheld medical scanning system of claim 14, wherein there are at least two sensors and the sensors extend in parallel.

16. A diagnostic and therapeutic device which comprises a system of claim 1 and a feedback loop to diagnose and provide treatment to a patient.

17. A method for locating and optionally removing a foreign body within a patient's body on or beneath the patient's skin prior to or after surgical exploration, removal, revision, or replacement, which comprises using a handheld medical scanning system of claim 1 to determine the location and/or size and/or depth of the foreign body.

18. The method of claim 17, wherein the location of the foreign body is marked on the patient's skin.

19. The method of claim 17, wherein a tool forming part of the medical system is used to extract the foreign body.

20. The method of claim 17, wherein a foreign body is located by a method applied at or near a surface of the patient's skin.

21. A handheld scanning device for determining the location of a foreign body in or beneath a patient's skin, which comprises
one or more sensors that each generate a magnetic or electromagnetic field and a signal reflecting the location of the foreign body;
a processor that receives and processes each signal and generates a signal to an operatively connected display; and
a display that receives the display signal and shows the location of the foreign object.

22. The handheld scanning device of claim 21, wherein there are two, three, or four longitudinally extending sensors arranged substantially parallel to one another.

23. The handheld scanning device of claim 22, wherein the device has a base and there are three sensors equidistantly positioned around the base.

24. The handheld scanning device of claim 21 which is capable of determining the location of the foreign object within the patient's body and of finding and removing the foreign object.

25. The handheld scanning device of claim 21 which is capable of determining the location of the foreign object within the patient's body, of marking the location of the foreign object, and of finding and removing the foreign object.

26. The handheld scanning device of claim 21 wherein the display shows the location and/or size and/or depth of the foreign object.
